(19) United States
(12) Patent Application Publication
(10) Pub. No.: US 2026/0337572 A1
(43) Pub. Date: Oct. 29, 2026

(54) METHOD OF CULTURING MUSCLE CELLS WITH PERFUSION BIOREACTOR

(71) Applicant: CELLULAR AGRICULTURE LTD., Llanelli (GB)

(72) Inventors: Marianne Jane Ellis, Corsham Wiltshire (GB); Illtud Llyr Dunsford, Llanelli Carmarthenshire (GB); Scott James Allan, Bath Somerset (GB)

(21) Appl. No.: 19/103,794

(22) PCT Filed: Aug. 17, 2023
(86) PCT No.: PCT/GB2023/052165

§ 371 Date: Feb. 13, 2025

(30) Foreign Application Priority Data
Aug. 18, 2022 (GB) 2212079.4

(51) Int. Cl. C12N 5/077; A23L 13/00

(52) U.S. Cl. CPC C12N 5/0658; A23L 13/03; C12N 2527/00

(57) ABSTRACT

Provided herein is a method of culturing muscles cells for a comestible product, the method seeding muscle cells onto the one or more porous hollow fibres; maintaining the perfusion module in an orientation wherein the one or more porous hollow fibres is at an angle between 0 to 90 from horizontal for a time period sufficient to allow initial attachment of the muscle cells to an outer surface of each of the one or more porous hollow fibres; rotating the perfusion module; connecting the perfusion module to a perfusion bioreactor system; and culturing the muscle cells attached to the outer surface of the porous hollow fibres. Also included are comestible products produced by such methods.

The present invention provides methods of culturing muscle cells. In particular, provided herein are methods of culturing muscles cells using a hollow fibre bioreactor (HFB). Also provided are comestible products including or formed from the cells produced from by the methods or the cells and hollow fibres used in the methods.

### BACKGROUND

There is growing interest in cultured meat as a protein alternative. Lab-scale production of cultured meat in its simplest form of muscle cells or co-cultures of muscle and fat cells has been achieved, however scaling-up the process to make a viable economic product is still challenging.

Challenges to be overcome include ethical sourcing of raw materials, reducing cost of cell culture media, increasing protein yield for the muscle cell culture, and within the bioprocess itself improved energy efficiency and resource utilisation and waste valorisation.

For increased process efficiencies and reduced environmental impact, bioreactors with higher cell densities allow a smaller culture volume thus reducing space requirements, labour requirements to set up and harvest the cells, and the amount of raw materials to manufacture them. Operating costs will be also lower as smaller bioreactors requiring less power and utilities.

Native skeletal muscle anatomy, consists of several arrays of uniaxial, striated myofibres in conjunction with fat cells, fibroblasts, capillaries and veins. A capillary is connected to most of the myofibres in a fascicle to provide blood perfusion, to provide the muscle cells with adequate oxygen and nutrients and also take away cell metabolism waste. This structure is well-replicated in a hollow fibre bioreactor (HFB) where the inlet media carries oxygen and nutrients, goes through the fibres and nourishes the cells and the perfused flow (permeate) and/or retentate carries out the wastes at an outlet.

Although the concept of a HFB seems to satisfy the targeted milestones to have higher cell density and a vascularised structure of whole-cut meat, practical challenges are yet to be overcome; such as:

Hollow fibre bioreactors are classified as a hydraulic bioreactor, meaning mixing is achieved via liquid flow rather than by mechanical mixing. This involves seeding the cells in a matrix with porous hollow fibres to allow cells to adhere to the hollow fibre surface where the medium may also circulate. A hollow fibre system offers the benefits of creating low shear stress, increased selection of which nutrients are transported, and is ideal for highly metabolic cell types. However, hollow fibre bioreactors are mostly limited to cell culture and do not support culturing tissue scaffolds.

Baba K, Sankai Y. Development of biomimetic system for scale up of cell spheroids—building blocks for cell transplantation. 2017 39th Annu Int Conf IEEE Eng Med Biol Soc. 2017. 10.1109/EMBC.2017.8037147, Yamamoto Y, Ito A, Jitsunobu H, Yamaguchi K, Kawabe Y, Mizumoto H, Kamihira M. Hollow Fiber Bioreactor Perfusion Culture System for Magnetic Force-Based Skeletal Muscle Tissue Engineering. J Chem Eng Japan. 2012; 45:348-54. https://doi.org/10.1252/jcej. 11we237, and Bettahalli N M S, Vicente J, Moroni L, Higuera G A, Van Blitterswijk C A, Wessling M, Stamatialis D F. Integration of hollow fiber membranes improves nutrient supply in three-dimensional tissue constructs. Acta Biomater [Internet]. Acta Materialia Inc.; 2011; 7:3312-24 all disclose the use of hollow fibres for culturing C2C12 cells in a perfusion bioreactor. However, these all disclose that the cells are attached to or maintained in a separate scaffold or matrix rather than attached to the hollow fibres. The hollow fibres are only used for supplying media to the cells and not for support or scaffolding purposes.

Bettahalli N M S, Steg H, Wessling M, Stamatialis D. Development of poly(l-lactic acid) hollow fiber membranes for artificial vasculature in tissue engineering scaffolds. J Memb Sci [Internet]. Elsevier B.V.; 2011; 371:117-26 discloses the use of a PLLA hollow fibre system to culture C2C12 cells. The fibres are used both for supplying media to the cells and as a scaffold for the cells to attach to. However, when media was supplied continuously the cell number decreased (i.e. the cells were not able to stay attached and grow in the bioreactor).

Luetchford, K. A.; Wung, N.; Argyle, I. S.; Storm S. P.; Weston S. D.; David Tosh D.; Ellis M. J (50%). Next generation in vitro liver model design: Combining a permeable polystyrene membrane with a transdifferentiated cell line. Journal of Membrane Science. 2018. 565 pp. 425-438 discloses the use of hollow fibres for culturing liver cells for an in vitro model. However, there is no suggestion of using such systems for scaled cell expansion or for muscle cells or how these methods may be adapted to suit muscle cells.

There is a need for improved systems and methods for culturing muscles.

There is a need for improved methods of culturing muscle cells for producing meat analogues.

There is also a need for improved methods for continuous culturing of muscle cells or providing increased yields of cells.

### BRIEF SUMMARY OF THE DISCLOSURE

The invention is based on the surprising finding that the use of the methods described herein provide high yields of muscle cells suitable for use in or production of meat analogues. For example, it has been found that maintaining the prefusion module at an angle of between about 0 to 90 degrees for a period of time allows for attachment of the cells to the hollow fibres. It has also been found that culturing cells at an angle of between about 0 to 90 degrees may allow for higher amounts of cells to be produced.

In one aspect of the invention there is provided a method of culturing muscles cells for a comestible product, the method comprising:
a) providing a perfusion module for a perfusion bioreactor system;
   the perfusion module comprising one or more porous hollow fibres comprising and outer surface and an internal lumen;
b) seeding muscle cells onto the one or more porous hollow fibres;
c) maintaining the perfusion module in an orientation wherein the one or more porous hollow fibres is at an angle between 0 to 90 from horizontal for a time period sufficient to allow initial attachment of the muscle cells to an outer surface of each of the one or more porous hollow fibres;
d) rotating the perfusion module;
e) connecting the perfusion module to a perfusion bioreactor system; and
f) culturing the muscle cells attached to the outer surface of each of the one or more porous hollow fibres under conditions suitable for proliferation and/or differentiation of the muscle cells.

In certain embodiments, the time period is at least 10 minutes.

In certain embodiments, the angle is between 0 and 30 degrees from horizontal. In certain embodiments, the angle is between 5 and 20 degrees from horizontal. In certain embodiments, the angle is between 8 and 10 degrees from horizontal. In some embodiments, the angle is about 9.2 degrees.

In certain embodiments, rotating comprises exerting a centrifugal force of between 0 and 50 N. For example, exerting a centrifugal force on the perfusion module and cells seeded therein.

In certain embodiments, rotating comprises continuously rotating. In certain embodiments, rotating comprises continuously rotating at a speed of between 0 and 30 rpm. In certain embodiments, rotating comprises continuously rotating at a speed of about 1 to 4 rpm. In certain embodiments, rotating comprises continuously rotating for at least 1 hour. In certain embodiments, rotating comprises continuously rotating for at least 3 hour. In certain embodiments, rotating comprises continuously rotating for about 3 to 5 hours.

In certain embodiments, rotating the perfusion module comprises rotating the perfusion module with an offset between the mid-point of the centreline of the perfusion module and the axis of rotation. In certain embodiments, the offset is greater than 0 mm. In certain embodiments, the offset is at least 10 mm. In certain embodiments, the offset is up to 500 mm. In certain embodiments, the offset is about 150 mm.

In certain embodiments, the attachment efficiency is at least 5% as measured by quantification of viable cells attached to the hollow fibres by direct or indirect measurement. In certain embodiments, the attachment efficiency is at least 10%, 20%, 30%, 40% or 50% as measured by quantification of viable cells attached to the hollow fibres by direct or indirect measurement In certain embodiments, the attachment efficiency is at least 55% as measured by quantification of viable cells attached to the hollow fibres by direct or indirect measurement.

In certain embodiments, rotating the perfusion module comprises intermittently rotating the perfusion module. In certain embodiments, rotating the perfusion module comprises rotating at a speed of between 0 and 30 rpm for about 1 second to about 5 minutes about every 10 seconds to about 30 minutes for a total time of at least 1 hour.

In certain embodiments, rotating the perfusion module comprises rotating at a speed of about 12 rpm for about 30 seconds about every 5 minutes.

In certain embodiments, rotating the perfusion module comprises rotating for at least three hours.

In certain embodiments, prior to seeding the muscle cells are:
(i) washed with a composition comprising a cell detachment agent;
   (ii) harvested in a culture media comprising a growth promotion agent;
   (iii) centrifuged to form a pellet; and
   (iv) resuspended in a culture media to form a cell suspension.

In certain embodiments, seeding the muscle cells comprises applying the cell suspension comprising the muscle cells to the perfusion module.

In certain embodiments, seeding the muscle cells comprises applying around at least 0.1-fold higher cell density than used for 2D tissue culture plastic. For example, 0.1 to 10 fold higher cell density.

In certain embodiments, seeding the muscles cells comprises applying at least 1000 cells/cm² to the perfusion module.

In certain embodiments, the one or more porous hollow fibres:
are hydrophilic;
are washable; and/or
are reusable.

In certain embodiments, the one or more porous hollow fibres comprise polystyrene.

In certain embodiments, the one or more porous hollow fibres are biodegradable and/or edible.

In certain embodiments, culturing comprises pumping culture media through the perfusion bioreactor system and the internal lumen of each of the one or more porous hollow fibres; optionally
wherein culturing further comprises maintaining the perfusion module is maintained in an orientation wherein the one or more porous hollow fibres is at an angle between 0 to 90 from horizontal. Optionally wherein the angle is between 0 and 30 degrees from horizontal; preferably between 5 and 20 degrees; more preferably between 8 and 10 degrees

In certain embodiments, culturing comprises pumping culture media through the perfusion bioreactor system and the internal lumen of each of the one or more porous hollow fibres at a rate of at least 10 μL/hour/hollow fibre.

In certain embodiments, culturing comprises maintaining the muscle cells at a temperature of at least 15 °C and/or 5% CO₂.

In certain embodiments, culturing comprises pumping culture media through the perfusion bioreactor system and perfusion module for at least 3 hours.

In certain embodiments, culturing comprises pumping a first culture media through the perfusion bioreactor system and the internal lumen of each of the one or more porous hollow fibres, optionally wherein the first culture media is a proliferation medium.

In certain embodiments, culturing further comprises pumping a second culture media through the perfusion bioreactor system and the internal lumen of each of the one or more porous hollow fibres, optionally wherein the second culture media is a differentiation medium; further optionally wherein the differentiation media is perfused through the bioreactor system and perfusion module for at least 3 hours.

In certain embodiments, the perfusion bioreactor system comprises apparatus for monitoring metabolite concentration and/or oxygen concentration of the culture media.

In certain embodiments, the method further comprises:
(g) harvesting the muscle cells from the perfusion module.

In certain embodiments, the harvested muscle cells are formed into a cultured meat product; or wherein the one or more hollow fibres are edible, and the harvested cells and the one or more porous hollow fibres are formed into a cultured meat product.

In certain embodiments, the muscle cells are derived from at least one comestible animal cell; optionally wherein the muscle cells comprise one or more of fibroblasts, skeletal muscle cells, smooth muscle cells, and/or myoblasts; further optionally wherein the muscle cells ae derived from one or more of non-human embryonic stem cells and/or pluripotent stem cells.

In another aspect of the invention there is provided a comestible product comprising muscle cells obtained by a method as described herein.

In another aspect of the invention there is provided a comestible product obtainable by the method as described herein.

In certain embodiments, the comestible product is a cultured meat product.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows the in vivo-like environment of a HFB. Cells are seeded onto the outside of the porous fibres. Media is delivered through the fibre lumen, mimicking a blood capillary. (A) Longitudinal section of a fibre (not to scale). (B) Cross section of a 3 fibre reactor; and

FIG. 2 shows an HFB module. (A) The dimensions of the module. The dimensions were chosen to fit 3 fibres. Different sizes can be manufactured and tailored for individual systems and fibres. (B) A photograph of the module with attached end cap and module connectors.

FIG. 3 shows module fabrication. (A) Fibres are cut to size and inserted into the module. (B) Fibres are glued into the module, allowed to dry. (C) The ends are cut flush with the glass.

FIG. 4 shows HFB system setup. Arrows indicate direction of media flow. A=feed tube. B=pump tube. E=HFB module. D=retentate tube with clamp. C=permeate tube.

FIG. 5 shows an indirect measure of metabolic activity of RSkMCs on tissue culture plastic (TCP) and hydrophilic polystyrene (PX40) flat sheet membranes. Metabolic activity assessed using a resazurin assay. Data represents mean±SD, n=1, N=6.

FIG. 6 shows relationship between midpoint of the (axial) centreline of a perfusion module to axis of rotation in determining offset.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

The methods described herein relate to the culturing of cells as described herein using a hollow fibre perfusion bioreactor.

Perfusion culturing refers to a continuous culturing method in which cells are either retained in the bioreactor or fed back into it. The cell culture medium perfused through the bioreactor thus contains no cells. Perfusion-based culturing methods may result in higher cell concentrations and product yields in the reactor while reducing the working volume, for example, in view of continuous stirred tank reactors methods and systems.

As used herein, the term “perfusion bioreactor” refers to a cell culture system in which the cell culture medium (e.g., a first cell culture medium, a second cell culture medium, a culture medium, a cell proliferation cell culture medium, and/or a cell differentiation cell culture medium) is continuously replaced with fresh media. Perfusion bioreactor systems may include means (e.g., an outlet, inlet, pump, or other such devices) for periodically or continuously withdrawing and adding substantially the same volume of replacement cell culture medium to the bioreactor. The addition of the replacement liquid culture can be performed substantially simultaneously with or immediately after the removal of the initial cell culture medium from the bioreactor. The means for removing the liquid culture from the bioreactor and for adding the replacement liquid culture may be a single device or system. For example, the means for removing and replacing (i.e. perfusing cell culture media) may be a peristaltic pump system.

A “bioreactor” may be any device or system that maintains a biologically active environment, for example, a chamber or vessel in which cells can be cultured. There are several different bioreactor types that differ in shape (e.g., cylindrical or otherwise), size (e.g., millilitres, litters to cubic meters), and materials (stainless steel, glass, plastic, etc.). Accordingly, the bioreactor is adapted to grow cells or tissue in cell culture. The bioreactor may be configured to receive hollow fibres as described herein. A hollow fibre perfusion bioreactor (HFB) includes a perfusion module (which may also be referred to as a bioreactor) with hollow fibres contained therein.

General HFBs and perfusion systems for use with a perfusion module as described herein, for example, include those described in Luetchford, K. A.; Wung, N.; Argyle, I. S.; Storm S. P.; Weston S. D.; David Tosh D.; Ellis M. J (50%). Next generation in vitro liver model design: Combining a permeable polystyrene membrane with a transdifferentiated cell line. Journal of Membrane Science. 2018. 565 pp. 425-438, and Wung, N., Acott, S. M., Tosh, D., Ellis, M. J. Hollow fibre membrane bioreactors for tissue engineering applications. Biotechnol Lett. 36 (12) 2357-2366 (2014).

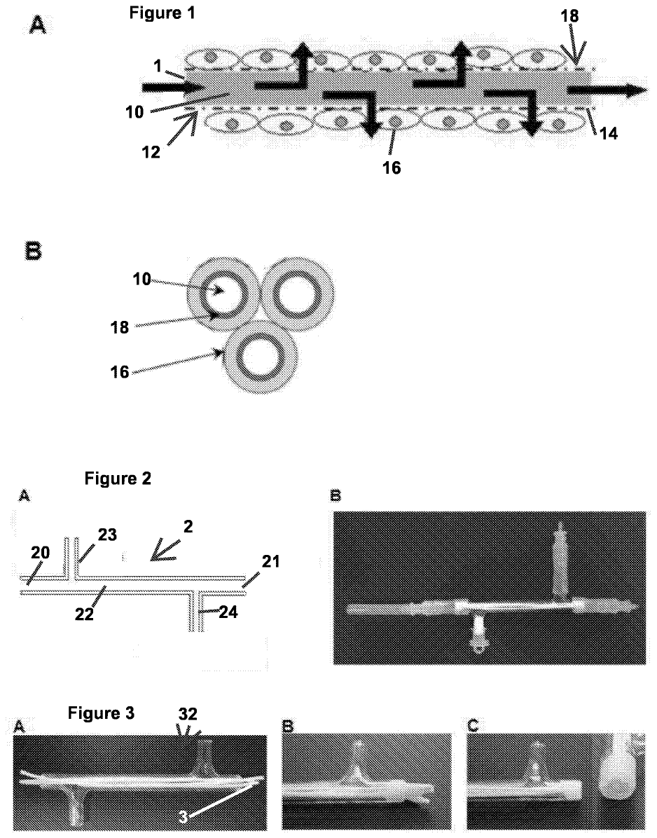

Typically a hollow fibre perfusion bioreactor system as used in the methods described herein includes a continuous perfusion system with a constant replenishment of nutrients and removal of waste. Medium circulates through a perfusion module, including porous hollow fibres. The interior of the hollow fibres may be termed the Intracapillary space (ICS) or internal lumen, and the exterior may be termed the extracapillary space (ECS). Cells grow in the ECS and adhere to the outside of the fibres (outer surface). The fibres may provide a large surface area for cell contact in a relatively compact system. A schematic diagram of the general concept of a hollow fibre system is shown in FIG. 1. As can be seen in FIG. 1, media (1) flows through the lumen (10) of a hollow fibre (12). Pores (14) in the hollow fibre allow for the transfer of nutrients to cells (16) which adhere to the outer surface (18) of the hollow fibre.

Typically, small molecular weight cellular nutrients and metabolic waste products can pass between the ICS and the ECS via the pores of the porous hollow fibres, whilst cells may be confined to the ECS. One or more “side” ports may be used to introduce large molecular weight nutrients and/or flow media or liquids into the ECS to provide defined shear rates to the cells. An additional side port may be used to collect fluid flowing through the ECS. HFBs offer an in vivo-like environment with the fibres mimicking blood capillaries and shielding the cells from the shear stresses associated with dynamic media delivery while allowing defined shear to be applied to cells via fluid flow through the side ports if desired. This creates a versatile culture system with superior mass transport in which high cell densities can be reached.

An example of a perfusion module is shown in FIGS. 2 and 3. In FIG. 2A a schematic of an example perfusion module is shown. The perfusion module (2) includes an inlet port (20) and an outlet port (21) located at each end of a channel (22). The example shown also includes a first side port (23) and a second outlet port (24). FIG. 3 shows a perfusion module with hollow fibres (3) inserted into the perfusion module channel (32).

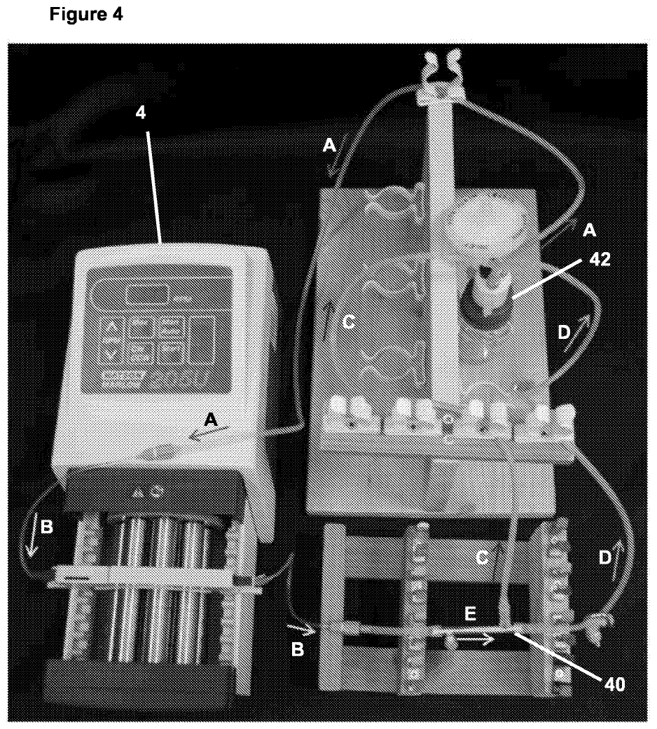

An example perfusion system setup is shown in FIG. 4. The system includes a pump system (4) connected to a perfusion module (40 and E). Culture media is fed from a reservoir (42) via a feed tube (A) to the pump system (4), which then pumps media to the perfusion module via pump tubing (B). The culture media flows through the perfusion module and exits from the lumens of the hollow fibres via retentate tubing (D) and media that has exchanged through the pores of the hollow fibres and into ECS via permeate tubing (C).

The pumping system may be any suitable pump. For example, the pump system may be a peristaltic pump system or vacuum pump system. The flow rate of culture media that is flowed through the perfusion module may be controlled by the pump system. The flow rate may be selected depending on the number of hollow fibres being used, the cells being cultured and/or the number of cells seeded onto the outer surface of the hollow fibres.

For example, culture media may be pumped at a rate of at least 1 μL/hour/hollow fibre. That is to say, for each hollow fibre used, the flow rate is 1 μL/hour. In some examples, the flow rate is at least 10 μL/hour/hollow fibre. For example, at least 10, 20, 30, 40, 50, 60, 70, 80, 90, 100, 200, 300, 400 μL/hour/hollow fibre. In some examples, the flow rate is from 100 to 300 μL/hour/hollow fibre. In some examples, the flow rate is between 240 to 280 μL/hour/hollow fibre. In some examples, the flow rate is about 267 μL/hour/hollow fibre.

The perfusion bioreactor system may further include monitoring apparatus. Monitoring apparatus refers to any apparatus suitable for determining and/or monitoring changes in one or more properties of cells being cultured and/or one or more properties of the perfused media after having flowed through the hollow fibres (retentate) and/or that has passed though the pores (permeate).

For example, the monitoring apparatus may be a system for monitoring nutrient and/or metabolite content of the permeate and/or retentate. As used herein, permeate refers to culture media that has passed through the pores of the hollow fibres (i.e. permeated through the hollow fibres). As sued herein, retentate refers to culture media that flows through the internal lumen of the hollow fibres (i.e. is retained within the hollow fibres). Nutrients that may be monitored may be any nutrient and/or metabolite that is included in the culture media used. For example, the nutrient and/or metabolite may be one or more of a saccharide (such as glucose), lactic acid (lactate), glutamine, glutamate, ammonia (ammonium ion), essential and non-essential amino acids, growth factors, albumin, attachment proteins, vitamins, oxygen, and or carbon dioxide or the like.

The concentrations of nutrients and/or metabolites may be compared to the initial concentration of the corresponding nutrients and/or metabolites in the culture media prior to culturing the cells or prior to being perfused through the hollow fibres. Changes in concentrations of nutrients and/or metabolites in the permeate and/or retentate in comparison to the initial cell culture media may provide information on the rate of proliferation, cell number and/or condition (e.g. viability and/or health) of the cells being cultured.

In some examples, systems for monitoring concentrations of nutrients and/or metabolites may be separate from the perfusion bioreactor system. For example, the methods may further include collecting a portion of the permeate and/or retentate at predetermined time points and determining the concentrations of nutrients and/or metabolites. In some examples, the perfusion bioreactor system may include a system for monitoring nutrients and/or metabolites that is connected directly to the perfusion bioreactor system.

Suitable methods for determining concentrations of nutrients and/or metabolites in culture media are well known in the field. For example, kits such as those provided by Megazyme are available that include instructions therein. For example, for nutrients and/or metabolites such as saccharides (such as glucose), lactic acid (lactate), glutamine, glutamate, ammonia (ammonium ion). Alternatively or additionally HPLC and/or GC-MS may be used to determine concentrations of essential and non-essential amino acids, growth factors, albumin, attachment proteins. For example, monitors include those available from PreSens for oxygen and carbon dioxide.

In some examples, the monitoring apparatus may include a system for monitoring a concentration of dissolved oxygen in the permeate and/or retentate. The concentrations of dissolved oxygen may be compared to the initial concentration of dissolved oxygen in the culture media prior to culturing the cells or prior to being perfused through the hollow fibres. Changes in concentrations of dissolved oxygen in the permeate and/or retentate in comparison to the initial cell culture media may provide information on the rate of proliferation, cell number and/or condition (e.g. viability and/or health) of the cells being cultured.

Systems for determining the concentration of dissolved oxygen in culture media are well known and include, for example, PreSens flow-through cell and PreSens Fibox 4 oxygen reader.

The cells seeded onto the hollow fibres include muscle cells. Muscle cells include those cells making up contractile tissue of animals or cells that can differentiate into muscle cells. Muscle cells are derived from the mesodermal layer of embryonic germ cells. Mature muscle cells contain contractile filaments that move past each other and change the size of the cell. They are classified as skeletal, cardiac, or smooth muscles. As used herein, the term “cells that can differentiate into muscle cells” refers to stem cells and muscle progenitor cells that can differentiate into muscle cells (e.g. mature muscle cells).

Muscle cells may include those cells normally found in muscle tissue, including smooth muscle cells, cardiac muscle cells, skeletal muscle cells (e.g., muscle fibres or myocytes, myoblasts, myotubes, etc.), and any combination thereof. Muscle cells may include myoblasts, myotubes, myofibrils, and/or satellite cells.

The cells may further include adipose or fat cells. Adipose or fat cells include any cell or group of cells composed in a fat tissue, including, for example, lipocytes, adipocytes, adipocyte precursors including, pre-adipocytes and mesenchymal stem cells.

The cells may be derived from any source animal. As the perfusion modules described herein may be for use in making comestible products, the cells may not be derived from a human. In some examples, the cells may be derived from bovine, ovine, equine, porcine, caprine, avian, fish, insect, crustaceans, cephalopod, mollusc and/or camelid animals. Preferably the cells may be derived from a bovine, porcine, avian and/or ovine animal. For example, the cells may be derived from a cow, pig, chicken, fish, squid, insect, oyster and/or sheep.

The cell culture medium that may be used in the methods described herein may be any suitable cell culture medium. The cell culture medium may be selected depending on the type of cell cells being cultured. Examples of culture mediums that may be used include minimal essential medium (MEM, Sigma, St. Louis, Mo); Dulbecco's modified Eagle medium (DMEM, Sigma); Ham F10 medium (Sigma); Cell culture media (HyClone, Logan, Utah); RPMI-1640 culture media (Sigma); and chemical-defined (CD) culture media (which are formulated for individual cell types), such as CD-CHO culture media (Invitrogen, Carlsbad, Calif). The culture solution described above can be supplemented with auxiliary components or contents as needed. This includes any component of the appropriate concentration or amount required or desired.

The culture medium described above can be supplemented with auxiliary components or contents as needed. The culture medium may include one or more additives such as antibiotics, proteins, amino acids and/or sugars.

“Medium” and “cell culture medium” refer to a nutrient source used for growing or maintaining cells. As is understood by a person of skill in the art, the nutrient source may contain components required by the cell for growth and/or survival or may contain components that aid in cell growth and/or survival. Vitamins, essential or non-essential amino acids, trace elements, and surfactants (e.g., poloxamers) are examples of medium components. Any media provided herein may also be supplemented with any one or more of insulin, plant hydrolysates and animal hydrolysates.

“Culturing” a cell refers to contacting a cell with a cell culture medium under conditions suitable for the viability and/or growth and/or proliferation of the cell.

Perfusing the cell culture medium may include perfusing a first culture media and then subsequently perfusing one or more second cell culture mediums. The first cell culture medium may be a cell culture medium that is for proliferating cells and may be referred to as proliferation medium.

Proliferation medium may be a medium comprising a source of nutrients, such as vitamins, minerals, carbon and energy sources, and other beneficial compounds that facilitate the biochemical and physiological processes occurring during expansion or proliferation of cells. The proliferation medium may comprise one or more carbon sources, vitamins, amino acids, and inorganic nutrients. Representative carbon sources include monosaccharides, disaccharides, and/or starches. For example, the proliferation medium may contain one or more carbohydrates such as sucrose, fructose, maltose, galactose, mannose, and lactose. The proliferation medium may also comprise amino acids. Suitable amino acids may include amino acids commonly found incorporated into proteins as well as amino acids not commonly found incorporated into proteins, such as arginosuccinate, citrulline, canavanine, ornithine, and D-stereoisomers. The proliferation medium may also comprise growth promotion agents, such as serum, for example, foetal bovine serum (FBS). Examples of other growth promotion agents include growth factors (e.g. recombinant growth factors), bovine ocular fluid, sericin protein, earthworm heat inactivated coelomic fluid. In some examples, the proliferation media may be a serum free culture media and may optionally include additional components depending on the cells being cultured. For example, serum free culture medias include those commercially available from ThermoFisher, Lonza Bioscience and Merck. The proliferation medium may also comprise antibiotics.

For example, the proliferation medium may be Dulbecco's Modified Eagle's Medium (DMEM), which may include 10% (V/V) filter sterilised foetal bovine serum and 1% (V/V) penicillin/streptomycin solution.

In some examples, such as when the cells are derived from an insect, the proliferation media may be a media such as Gibco insect media available from ThermoFisher.

The cells may be maintained and cultured in proliferation medium for at least 3 hours. For example at least 3, 4, 5, 6, 7, 8, 9, 10, 11, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 125, 130, 135, 140, 145 or 150 hours. In some examples, the cells may be maintained in proliferation media continuously.

The cell culture media may be changed to a second cell culture medium. The second cell culture medium may be a differentiation medium. Differentiation medium refers to a medium designed to support the differentiation of cells, that is, supporting the process of a cell changing from one cell type to another. The differentiation medium may include one or more amino acids, antibiotics, vitamins, salts, minerals, or lipids. The differentiation medium may include at least one carbon source, such as a sugar. For example, glucose. The differentiation medium may include one or more proteins, amino acids or other additional acids. In some examples, the differentiation media includes one or more growth promotion agents, such as serum, for example, foetal bovine serum or horse serum. Examples of other growth promotion agents include growth factors (e.g. recombinant growth factors), bovine ocular fluid, sericin protein, earthworm heat inactivated coelomic fluid. In some examples, the differentiation media may be a serum free culture media and may optionally include additional components depending on the cells being cultured. For example, serum free culture medias include those commercially available from ThermoFisher, Lonza Bioscience and Merck. In some examples, the differentiation medium may be high-glucose DMEM (97%) supplemented with 2% horse serum and 1% penicillin/streptomycin solution.

The cells may be maintained and cultured in differentiation medium for at least 3 hours. For example at least 3, 4, 5, 6, 7, 8, 9, 10, 11, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 125, 130, 135, 140, 145 or 150 hours.

In some examples, the culture media may be changed a number of times. For example, the cells may first be cultured in proliferation media; then, the culture media is switched to differentiation media. The differentiation media may then be switched to proliferation media. The proliferation media may be the same as before or may contain different components and/or concentrations of components to the initial proliferation media.

In some examples, the cells may be continuously cultured. That is to say that the cells are maintained in one or more culture mediums as described herein. The cells may be cultured for a first time period (e.g. at least 3 hours or more) in a first media, then for a second time period (e.g. at least 3 hours or more) in a second media and then cultured in a further media and continuously cultured with any number of changes of the culture media so that cells can be constantly proliferated and/or differentiated. In such examples, the cells may be harvested at predetermined cell densities or time points in order to avoid overcrowding, reduced viability and/or cell death.

The cell culturing conditions (e.g. for either or both proliferation or differentiation) may be selected depending on the cell type and/or cell source. In some examples, cells may be cultured (e.g. proliferated and/or differentiated) at a temperature of at least 15 °C. In some examples, the cells may be cultured at a temperature of at least 15 °C, 16 °C, 17 °C, 18 °C, 19 °C, 20 °C, 21 °C, 22 °C, 23 °C, 24 °C, 25 °C, 26 °C, 27 °C, 28 °C, 29 °C, 30 °C, 31 °C, 32 °C, 33 °C, 34 °C, 35 °C, 36 °C, or 37 °C.

For example, for culturing of insect cells, the cells may be cultured at a temperature of from 15 °C to 32 °C.

For example, for culturing mammal cells, the cells may be cultured at a temperature of 37 °C.

For example, for marine animal cells (such as crustaceans, fish or mollusc cells), cells may be cultured at a temperature of from 15 °C to 32 °C. In some examples, marine animal cells may be cultured at a temperature between 15 °C to 30 °C.

The cells may be cultured in defined atmospheric conditions. For example, the cells may be cultured in an atmosphere that has predetermined humidity and/or gas concentration. For example, cells may be cultured in an atmosphere including at least 5% CO₂.

Polymers that may be used to form the hollow fibres may be any polymer suitable for culturing and/or maintenance of cells. Suitable polymers include biodegradable polymers. The polymer may be a biocompatible polymer. Biodegradable polymers are any polymers that may be broken down by biological systems, such as polymers that can be broken down into harmless products by the action of living organisms. Biocompatible polymers are, along with any metabolites or degradation products thereof, generally nontoxic to cells or to a recipient (such as a human or animal) and do not cause any significant adverse effects to cells or a recipient at concentrations resulting from the degradation of the polymers. Generally speaking, biocompatible polymers are polymers that do not elicit result in negative effects on cell health or in a recipient. As one use for the hollow fibres described herein is the production of comestible products, biocompatible and/or biodegradable polymers may be advantageous if the fibres or part thereof is consumed (for example, ingested) by a person.

Biodegradable polymers include linear aliphatic polyesters such as polylactic acid, polyglycolic acid, polycaprolactone, polyhydroxybutyrate, polyhydroxyvalerate and their copolymers within the aliphatic polyester family such as poly(lactic-co-glycolic acid) and poly(glycolic acid-co-caprolactone); copolymers of linear aliphatic polyesters and other polymers such as poly(glycolic acid-co-trimethylene carbonate) copolymers, poly(lactic acid-co-lysine) copolymers, tyrosine-based polyarylates or polyiminocarbonates or polycarbonates, poly(lactide-urethane) and poly(ester-amide) polymers; polyanhydrides such as poly(sebacic anhydride); polyorthoesters such as 3,9-diethyidiene-2,4,8,10-tetraoxaspiro-5,5-undecane based polymers; poly(ester-ether) such as poly-p-dioxanone; polyamides, poly(amide-enamines) and poly(amido amine) dendrimers; and phosphorus-based polymers such as polyphosphazene and poly [bis(carboxy-lactophenoxy)]phosphazene.

The polymers may be edible polymers. Edible polymers refers to any polymer that is acceptable for use in an edible product.

Examples of edible polymers include polyvinyl alcohol, carboxyvinyl polymer, hydroxypropylmethylcellulose, hydroxyethylcellulose, methylcellulose, ethylcellulose, low-substituted hydroxypropylcellulose, crystalline cellulose, carboxymethylcellulose sodium, a synthetic polymer compound such as carboxymethylcellulose calcium, carboxymethylcellulose and carboxymethylstarch sodium, sodium alginate, dextran, casein, pullulan, pectin, guar gum, xanthan gum, tragacanth gum, acacia gum, zein, gelatin, chitin and chitosan, silk, fibrin and polymer compounds obtained from natural products such as starch or soybean.

The use of an edible polymer may provide hollow fibres and products, including the hollow fibres which are edible. For example, a cell culture grown on the hollow fibres may provide for an edible product that does not require the removal of the hollow fibres before consumption.

The hollow fibres may be a digestible polymer. “Digestible” refers to a material that, when eaten by a subject, can be broken down into compounds that can be absorbed and used as nutrients or eliminated by the subject's body. Digestible polymers include BCS, polylactic acids (PLAs), synthetic polyamides, polycarbonates, polyisocyanurates (PIRs), polyurethanes, polyethers, proteins, polysaccharides (such as starches), polylactones, polylactams or glycols.

In some examples, the hollow fibres include poly(lactic-co-glycolic acid) (PLGA). For example, the hollow fibres may include 10% PLGA.

In some examples, the hollow fibres include a reusable polymer. A reusable polymer refers to a polymer that does not degrade over time or due to use in culturing cells. Reusable polymers may provide a perfusion module that can be washed and used multiple times. In some examples, the hollow fibres are hydrophobic. In some examples, the hollow fibres are washable. Washable refers to hollow fibres that can be washed and/or sterilised without sustaining damage or loss of function. Hollow fibres with such properties may reduce waste and costs.

For example, the hollow fibres may be made from polystyrene.

Hollow fibres may be solid or semi-solid substrates having openings or apertures (pores), which allow components of cell culture media, such as metabolites, nutrients and gases (e.g. oxygen), to be delivered to cells attached to the outer surfaces of the hollow fibres. Hollow fibres described herein allow the growth of cells on the outer surface of the hollow fibres. Thus the hollow fibres described herein act as a 3-dimensional matrix that allows for the culture and maintenance of cells in a 3-dimensional architecture.

The hollow fibres may have a Young's modulus of at least 1000 Pa. In some examples, the hollow fibres have a Young's modulus from 1000 Pa to 1000000000 Pa. In some examples, the hollow fibres have a Young's modulus between 8000 Pa to about 20000 Pa.

For example, the hollow fibres may have a Young's modulus of around 115 MPa.

The pores of the porous polymer sheets may have an average pore diameter suitable for allowing infiltration and support cells within the polymer sheet. The pores may have an average diameter from at least 0.001 μm to 100 μm. For example, the porous scaffold may have pores with an average pore diameter of around 0.001, 0.002, 0.003, 0.004, 0.005, 0.006, 0.007, 0.008, 0.009, 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000 μm. The pores may have an average diameter from 0.001 μm to 5 μm.

In some examples, the pores may have an average diameter of around 2.5 μm. In some examples, the pores may have an average pore diameter of around 5 μm.

Average pore size may be determined using optical methods such as scanning electron microscopy (SEM), atomic force microscopy (AFM), computed tomography methods and/or transmission electron microscopy (TEM). Other methods that may be used include X-ray refraction methods, imbibition methods, mercury injection methods, and gas expansion methods.

The average pore size and porosity (or density of pores) may affect the penetration of cells into the scaffold and define the spatial distribution of cells within the 3D matrix of the scaffold. In addition, average pore size and porosity may affect the flow resistance, the transportation of nutrients, and/or the excretion of waste products from cells cultured thereon and/or therein.

The lumens of each hollow fibre act as conduits to transport cell culture medium to the cells located on the outer surface of the hollow fibres, as well as transporting waste products from the cells. The density of pores on each hollow fibre may be at least 1 pore/mm². For example, at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300 pores/mm².

In some examples, the pore density may be around 210 pores/mm².

The lumens of each hollow fibre may have an internal diameter from 1 μm to 1000 μm. The internal diameter refers to the diameter measured between the inner surface of the lumen. For example, the inner lumen diameter may be about 500 μm.

In some examples, the inner lumen diameter is from about 600 to about 700 μm. In some examples, the inner lumen diameter may be around 630 to 660 μm. In some examples, the inner lumen diameter is from about 50 to about 200 μm.

Each of the hollow fibres may have an outer diameter of between about 12 μm and 2000 μm.

In some examples, each of the hollow fibres may have an outer diameter from 500 to about 1000 μm. For example, 850 to 1000 μm. For example, the outer diameter of each hollow fibre may have an outer diameter of from about 900 to about 950 μm.

The length of each hollow fibre may be at least 1 cm. In some examples, the length of each hollow fibre may be at most 10 m. In some examples, each hollow fibre has a length from 1 cm to 10 m. In some examples, each hollow fibre has a length from 3 cm to 5 m. In some examples, each hollow fibre has a length from 10 cm to 2 m.

The hollow fibres may be produced by any method, many of which are known in the field. For example, melting spinning, solution spinning, wet spinning, gel spinning, dry-wet spinning, liquid crystal spinning, dispersion spinning, reaction spinning and electrospinning.

In some examples, the hollow fibres may be fibres as described in Luetchford, Kim A., et al. “Next generation in vitro liver model design: Combining a permeable polystyrene membrane with a transdifferentiated cell line.” Journal of membrane science 565 (2018): 425-438, which is incorporated herein in its entirety.

In some examples, the perfusion module may include at least 1 hollow fibre. For example, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 100, 150, 200, 250, 300, 400, 500, 600, 800, 1000 1000, 2000, 3000, 4000, 5000, 6000, 7000, 8000, 9000, 10000, 15000, 20000, 25000, 30000, 35000, 40000, 45000, 50000 or more. In some examples, the perfusion module may include from 1 to 50000 hollow fibres.

The methods described herein may include initial sterilisation and/or wash steps. For example, the perfusion module, tubing and other components of the perfusion system may be sterilised. Depending on the material of each component, sterilisation may be by autoclaving and/or by application of a sterilisation composition, for example, application of 70% ethanol. For example, the perfusion module without hollow fibres included and reservoir may be autoclaved prior to being connected to the perfusion system.

After connection of the perfusion module to the perfusion reactor system, ethanol may be pumped through the system in order to sterilise the component parts of the system. The sterilisation composition may be pumped (or perfused) through the system for at least 30 minutes. For example, at least 30, 40, 50, 60 minutes or more.

After sterilisation, the methods may include a wash step. The wash step may include removing any sterilisation composition from the system. For example, by draining the sterilisation solution from the system. The wash step may then further include pumping a culture media through the system so as to saturate the system and contact all component parts with the culture media. The cell culture may then be maintained in the system. In some examples, the culture media used for the wash step may be replaced with a further culture media as described herein. For example, a proliferation media as described herein.

The methods described herein include a step of seeding cells onto the hollow fibres in the perfusion module. Prior to seeding, the cells may be pre-cultured. For example, the cells may be pre-cultured outside of the perfusion system to form a cell suspension (seeding culture.

The cell suspension may be formed by first washing cells in a buffer, such as phosphate buffered saline (PBS). The buffer may include additional components such as a cell detachment agent. A cell detachment agent may be, for example, an enzyme such as trypsin, trypLE or nattokinase. In some examples, the cell detachment agent may be a composition, for example, acctuase. For example, the buffer may include at least 0.05% trypsin. In some examples, the buffer includes about 0.25% trypsin. Additional components may then be added to the buffer including the cells. For example, EDTA may be added to the buffer and cells. The buffer and cells may then be incubated for a period of time. For example, at least 3 minutes. For example, about 5 minutes.

Once incubated, a portion of the cells in the buffer may be mixed with a volume of culture media that includes a growth promotion agent, such as serum, for example, foetal bovine serum (FBS). The serum may help to neutralise the cell detachment agent. Examples of other growth promotion agents include growth factors (e.g. recombinant growth factors), bovine ocular fluid, sericin protein, earthworm heat inactivated coelomic fluid. In some examples, the culture media may be a serum free culture media and may optionally include additional components depending on the cells being cultured. For example, serum free culture medias include those commercially available from ThermoFisher, Lonza Bioscience and Merck. The number of cells in the volume of the culture media may then be determined. For example, using a cell counter or other suitable methods.

After determining the number of cells in the volume of culture media, the volume of culture media may be centrifuged to pellet the cells therein. The cell pellet may then be resuspended in a culture media, for example, a proliferation media as described herein, to form the cell suspension.

The cell suspension may then be applied to the perfusion module and the hollow fibres therein. In some examples, the volume applied to the perfusion module may be a volume that at least partially fills the extra-capillary space (ECS). The remaining volume of the perfusion module may be filled with further culture media (not including cells). In some examples, the volume of cell suspension may be less than the ECS. In some examples, the cell suspension may be continuously circulated through the perfusion module and therefore continually contacted with the hollow fibres. Continuously circulating the cell suspension may allow for increased seeding efficiency.

In some examples, the number of cells to be applied to the perfusion module may be determined by the outer surface area of the hollow fibres. For example, at least about 1000 cells/cm² may be applied to the perfusion module. In some examples, around 25,000 cells/cm² may be applied to the perfusion module. For example, cells may be added to each square centimetre of each hollow fibre at a concentration of at least 100 cells/cm². Cells may be added to each square centimetre of the outer surface area of all of the hollow fibres at a concentration from 100 cells/cm² up to 500,000 cells/cm² or more. For example, at least 100 cells/cm², 200 cells/cm², 300 cells/cm², 400 cells/cm², 500 cells/cm², 600 cells/cm², 700 cells/cm², 800 cells/cm², 900 cells/cm², 1000 cells/cm², 1500 cells/cm², 2000 cells/cm², 2500 cells/cm², 3000 cells/cm², 3500 cells/cm², 4000 cells/cm², 4500 cells/cm², 5000 cells/cm², 5500 cells/cm², 6000 cells/cm², 6500 cells/cm², 7000 cells/cm², 7500 cells/cm², 8000 cells/cm², 8500 cells/cm², 9000 cells/cm², 9500 cells/cm², 10000 cells/cm², 10500 cells/cm², 11000 cells/cm², 11500 cells/cm², 12000 cells/cm², 12500 cells/cm², 13000 cells/cm², 13500 cells/cm², 14000 cells/cm², 14500 cells/cm², 15000 cells/cm², 15500 cells/cm², 16000 cells/cm², 16500 cells/cm², 17000 cells/cm², 17500 cells/cm², 18000 cells/cm², 18500 cells/cm², 19000 cells/cm², 19500 cells/cm², 20000 cells/cm², 20500 cells/cm², 21000 cells/cm², 21500 cells/cm², 22000 cells/cm², 22500 cells/cm², 23000 cells/cm², 23500 cells/cm², 24000 cells/cm², 24500 cells/cm², 25000 cells/cm², 25500 cells/cm², 26000 cells/cm², 26500 cells/cm², 27000 cells/cm², 27500 cells/cm², 28000 cells/cm², 28500 cells/cm², 29000 cells/cm², 29500 cells/cm², 30000 cells/cm², 30500 cells/cm², 31000 cells/cm², 31500 cells/cm², 32000 cells/cm², 32500 cells/cm², 33000 cells/cm², 33500 cells/cm², 34000 cells/cm², 34500 cells/cm², 35000 cells/cm², 35500 cells/cm², 36000 cells/cm², 36500 cells/cm², 37000 cells/cm², 37500 cells/cm², 38000 cells/cm², 38500 cells/cm², 39000 cells/cm², 39500 cells/cm², 40000 cells/cm², 40500 cells/cm², 41000 cells/cm², 41500 cells/cm², 42000 cells/cm², 42500 cells/cm², 43000 cells/cm², 43500 cells/cm², 44000 cells/cm², 44500 cells/cm², 45000 cells/cm², 45500 cells/cm², 46000 cells/cm², 46500 cells/cm², 47000 cells/cm², 47500 cells/cm², 48000 cells/cm², 48500 cells/cm², 49000 cells/cm², 49500 cells/cm², 50000 cells/cm², 50500 cells/cm², 51000 cells/cm², 51500 cells/cm², 52000 cells/cm², 52500 cells/cm², 53000 cells/cm², 53500 cells/cm², 54000 cells/cm², 54500 cells/cm², 55000 cells/cm², 55500 cells/cm², 56000 cells/cm², 56500 cells/cm², 57000 cells/cm², 57500 cells/cm², 58000 cells/cm², 58500 cells/cm², 59000 cells/cm², 59500 cells/cm², 60000 cells/cm², 70000 cells/cm², 80000 cells/cm², 90000 cells/cm², or 100000 cells/cm², 200000 cells/cm², 300000 cells/cm², 400000 cells/cm², 500000 cells/cm², 1,000,000 cells/cm² or more.

In some examples, the number of cells may be determined based on the number of cells used or seeding a 2-dimensional cell culture system. For example, the number of cells applied may be at least 0.1-fold greater than that used to seed cells onto a flat sheet (2D) cell culture system made of the same material as the hollow fibres. For example, 0.1, 0.2, 0.3, 0.4, 0.5, 0.6, 0.7, 0.8, 0.9. 1, 1.5, 2, 2.5, 3, 3.5, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 fold greater than that used to seed cells onto a flat sheet (2D) cell culture system made of the same material as the hollow fibres.

After application of the cells to the perfusion module, the perfusion module may be maintained at an angle from 0 to 90° from horizontal for a time period sufficient to allow the cells to attach or adhere to the outer surface of the hollow fibres. Maintaining the perfusion module at an angle as described herein may allow for the initial attachment of the cells and thus avoid uneven distribution of cells on the outer surfaces of the hollow fibres.

In some examples, the angle is about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90° from horizontal.

In some example, the module is maintained at an angle of between 0 and 30 degrees from horizontal. For example, between 5 and 20 degrees from horizontal. For example, between 8 and 10 degrees from horizontal. For example, between 8.5 and 9.5 degrees from horizontal. In some examples, the angle is about 9.2 degrees. That is to say that the hollow fibres in the module are maintained at the selected angle. The hollow fibres may be any shape and the angle may be defined in relation to an axis through the hollow fibres. For example, for a tubular substantially straight hollow fibre, the angle from horizontal may be taken as the angle between the horizontal plane and the axis running longitudinally through the hollow fibre (i.e. the axis is the same as the lumen of the hollow fibre). In some cases, the hollow fibre may be spiral, arcuate or non-linear. In such cases, the angle may be defined by an offset from the plane generally parallel to the horizontal plane. That is to say that when the perfusion module is placed in the horizontal plane, the perfusion module is then offset to the horizontal plane and ergo offsetting the fibres therein from the horizontal plane. It will be understood that the hollow fibres may not be uniform in shape and as such, reference to an angle from horizontal refers to an angle taken from a specific point of the perfusion module or hollow fibres (i.e. the centre point of the longitudinal axis running through the perfusion module or hollow fibres) and as such the angle may not be the same for all parts of the hollow fibres.

In some examples, the perfusion module may be maintained at an angle as described herein for a time period of at least 10 minutes. In some examples, the perfusion module may be maintained at the angle for the entirety of the incubation period and/or culturing (proliferation and/or differentiation).

After maintaining the perfusion module, the perfusion module, including the cells seeded therein, may then be cultured (incubated) for a time period to allow for further attachment and initial growth of the cells. The perfusion module and cells may be cultured (incubated) for a time of at least 2 hours. For example, at least 2, 3, 4, 5, 6, 7, 8, 9, 10 or more hours. In some examples, the perfusion module and cells may be cultured (incubated) for a time of about 3 hours. The perfusion module and cells may be cultured (incubated) under conditions (i.e. temperature and atmosphere) suitable to sustain viable cells and/or allow growth of the cells. For example, for mammalian cells, the perfusion module and cells may be cultured (incubated) at 37 °C in an atmosphere including 5% CO₂.

The perfusion module may be rotated during the initial attachment stage (seeding) and/or incubation time. In some examples, the perfusion module and cells are rotated intermittently. In some examples, the perfusion module and cells are rotated at a speed of between 0 to 30 rpm. In some examples, the perfusion module and cells are rotated at a speed of about 12 rpm. In some examples, the perfusion module and cells are rotated for 1 second to about 30 minutes every 10 seconds to 30 minutes. In some examples, the perfusion module and cells are rotated for about 30 seconds every 5 minutes. That is to say that the perfusion module is rotated for a period of 30 seconds and then remains stationary for 5 minutes before being rotated again. The perfusion module may be rotated for at least 1 hour. For example, at least 1, 2, 3, 4 or 5 hours.

In some examples, the perfusion module may be rotated continuously. The perfusion module may be continuously rotated at a speed of between 0 and 30 rpm. In some examples, the perfusion module is continuously rotated at a speed between 0 and 4 rpm. In some examples, the perfusion module is continuously rotated for at least 3 hours. For example, about 3.5 to about 5 hours.

Rotation of the perfusion module and cells may be performed by any suitable means. For example, using a tube rotator such as a MACSmix™ available from Miltenyi Biotech.

Rotation of the perfusion module and cells may be used to exert a centrifugal force on the cells. As such, the centrifugal force may be more than 0.001 N. For example, the centrifugal force may be at least 0.001 N. In some examples, the centrifugal force may be between 0 and 50 N. The application of centrifugal force may help to move the cells through the perfusion module and bring the cells into contact with the hollow fibres. Without being bound by theory, this may help to improve the attachment efficiency of the cells to the hollow fibres. Intermittent rotation of the module may help introduce motive centrifugal forces to free-floating cells which may improve mixing. The increased mixing may help provide an even distribution of cells across hollow fibre surfaces upon contact and attachment.

The force applied by rotation may be affected by the positioning of the perfusion module in relation to the axis of rotation. For example, the perfusion module may be positioned so as to have an offset between the mid-point of the axial centreline of the perfusion module and the axis of rotation. The centreline of the perfusion module refers to the plane running axially through the centre of perfusion module longitudinally as shown by the cross in FIG. 6. The mid-point of the centre line refers to the distance halfway along the centreline. Providing an offset in combination with the rotation described above may help to mix the cells within the perfusion module. Without being bound by theory, the mixing imparted by the offset and rotation may increase the surface area of the hollow fibres that is contacted by the cells and may therefore improve the attachment efficiency of the cells to the hollow fibres

Maintaining the perfusion module and/or rotation (intermittent or continuous) may help improve cell attachment and/or increase the number of viable of cells (i.e. viable cells attached to the hollow fibres). For example, increase the attachment efficiency in comparison to methods not including a maintaining step and/or rotation as described herein by at least 5%, 10%, 15%, 20%, 25%, 30%, 35%, 40%, 45%, 50%, 55%, 60%, 65%, 70%, 75%, 80%, 85%, 90%, 95%, 100% or more.

Cell viability may be measured using any suitable means known in the field. For example, cell viability may be measured by direct measurement methods. Direct, measurement methods include attaching the cells to the hollow fibres. None attached cells are then discarded, for example, by flushing the perfusion module with additional culture media. Prior to culturing (i.e. proliferation or differentiation) the fibres including attached cells are removed and the cells attached to the fibres are removed from the fibres (i.e. detached) by known means. For example using a detachment agent. The number of viable cells that have been detached from the fibres may then be determined. The number of viable cells may be determined by cell counting methods such as flow cytometry or FACS. For example, using a cell counter such as Chemometec NC-200. The total number of attached cells and/or the total number of viable cells may be used to determine an attachment efficiency.

In some examples, cell viability after attachment may be measured by indirect measurements methods. For example, the perfusion module may be drained of growth media and the total cells in the drained growth media are quantified (including non-viable and viable cells) using cell counting methods as described above. The attachment efficiency is then calculated by the equation: Indirect attachment efficiency=(inoculated cells-removed during drain post-seed)/inoculated cells (as %).

After rotating the perfusion module and cells, the perfusion module is connected to the perfusion system, for example, by connecting the tubing to each end of the perfusion module and any sideports that may be included.

Cell culture media as described herein may then be perfused (pumped) through the perfusion system and the lumens of the hollow fibres. The cell culture media may be a proliferation media as described herein. The culture media may be pumped from either end of the perfusion module. In some examples, the perfusion module is maintained at an angle from 0 to 90°. For example, about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90° from horizontal. Therefore, culture media may be pumped from the lower or upper end to the opposing end. In some examples, culturing includes maintaining the perfusion module and the hollow fibres with cells attached thereto at an angle of 0° from horizontal.

The cells may be cultured under suitable conditions for proliferation of the cells. For example, using the conditions as described herein, which may be selected depending on the type of cells being cultured. For example, for mammalian cells may be cultured (incubated) at 37 °C in an atmosphere including 5% CO².

In some examples, the culture media may be changed to a second culture media. For example, the culture media may be changed from a proliferation media to a differentiation media as described herein. After culturing in the differentiation media, cells may be striated. The cells may form myotubes. The cells may form striated myotubules along each channel. Without being bound by theory, the mechanical force from sheer stresses caused by fluid flowing in the ECS and/or the lumens may help the striation of the cells.

The second culture media may also be changed to a further media. The further media may be the same as the first culture media (e.g. switching from a differentiation media to a proliferation media) or may be a different culture media.

The culture media may be switched from a first, second or further media any number of times. In some examples, the culturing conditions may be changed depending on the media being perfused through the system.

The cells may be cultured in any of the first, second and/or third culture media for at least 3 hours. In some examples, cell may be cultured for at least 1 day. For example, 1, 2, 3, 4, 5, 6, or more days. In some examples, the cells are continuously cultured. This may provide a system that provides a high number of cells.

After culturing the cells, the cells may be harvested from the perfusion module. Cells may be harvested by removing the cells from the hollow fibres. In some examples, when the hollow fibres are formed from edible, biodegradable or digestible polymers, the cells may be harvested attached to the hollow fibres, for example, by extracting the hollow fibres from the perfusion module with the cells attached thereto.

The cells may be harvested when the cells reach a confluence of at least 60%. In some examples, cells may be harvested after proliferation and/or differentiation, for example, after culturing in proliferation media for a time period as described herein. If culturing includes the use of differentiation culture media, then cells may be harvested when the cells reach a predetermined protein density.

The harvested cells or the harvested cells and hollow fibres may be used to produce a comestible product. For example, the cells and hollow fibres may be formed into a meat analogue.

A meat analogue (which may also be referred to as cultured or in vitro meat) refers to a food product that is not produced by the slaughter of an animal but has structure, texture, aesthetic qualities, and/or other properties comparable or similar to those of slaughtered animal meat, including livestock (e.g., beef, pork), game (e.g., venison), poultry (e.g., chicken, turkey, duck), and/or fish or seafood substitutes/analogues. The term refers to uncooked, cooking, and cooked meat-like food products.

The cells or cells and hollow fibres may be configured to mimic the taste, texture, size, shape, and/or topography of a traditional slaughtered meat. For example, multiple sets of harvested cells or cells and hollow fibres cells may be combined in order to form a structure similar to a cut of meat or a portion-sized product. For example, bound together or compressed together. For example, harvested cells or cells and hollow fibres may be bound together by an edible adhesive such as transglutaminase.

The harvested cells or cells and hollow fibres may have further agents added in order to make the sensory properties, such as texture, taste, smell and visual properties, more similar to a meat. For example, one or more of fats, texturisers, bulking agents, thickeners, preservatives, flavour enhancers, antimicrobial agents, pH modulators, desiccants, vitamins, minerals, metals, salts, sweeteners, curing or pickling agents, colouring agents, or any combination thereof may be added to the cells or cells and hollow fibres. Additional agents may be dispersed through the cells or cells and hollow fibres via the lumens of the hollow fibres.

As such, also provided herein is a meat analogue including cells or cells and hollow fibres produced the methods as described herein.

Any meat analogue produced may have the dimensions of a whole cut of meat. For example, it may have at least one dimension (i.e. at least one of length, width or thickness) that is at least 10 cm. The thickness or diameter of such a meat analogue may be up to 50 cm.

When the hollow fibres are not edible, the cells may be removed from the hollow fibres. That is to say that the cells may be recovered from the hollow fibres. The cells may be recovered by applying a cell-support specific agent to the hollow fibres. A cell detachment agent may be any agent that is capable of detaching the cells from the hollow fibres. For example, the cell detachment agent may be an enzyme. For example, trypsin, trypLE or nattokinase. In specific examples, the cell detachment agent is nattokinase. Nattokinase is a fibrin-specific enzyme derived from fermented soybeans. The nattokinase may be food-grade nattokinase.

The cell detachment agent may be added to the hollow fibres that have been manipulated to be a flat sheet, for example, unrolled or may be applied to intact hollow fibres. The cell detachment agent may be applied at a concentration of at least 10 mg/ml. For example, the cell detachment agent may be applied at a concentration of at least 10, 20, 30, 40, 50, 60 or 70 mg/ml.

When the cell detachment agent has been applied, the cells are removed from the hollow fibres and suspended in a composition including the cell detachment agent. The recovered cells may then be separated from the composition, for example, by centrifugation or other known methods.

The recovered cells may then be used to produce a comestible product. The cells may be processed into a product such as a meat analogue. For example, the cells may be subjected to similar processes as used for producing products such as sausages or processed meats products, for example, reconstituted meats such as baloney. For example, the cells may be emulsified, ground, or minced and then formed into a product that resembles a cut of meat or a meat product. For example, processed cells may be moulded or shaped using any known methods. The cells may have agents added to help processing, such as fats, binders, or texturisers added. The cells may also have additional agents added in order to make the sensory properties, such as texture, taste, smell and visual properties, more similar to a meat. For example, one or more of fats, texturisers, bulking agents, thickeners, preservatives, flavour enhancers, antimicrobial agents, pH modulators, desiccants, vitamins, minerals, sweeteners, salts, metals, curing or pickling agents, colouring agents, or any combination thereof may be added to the cells.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Example 1—HFB Setup for Culture Muscle Cells

### 1. Fibers

1.98 Manufacture the fibers by phase inversion spin casting (spinning). Details of this method can be found in [4] and [5].

NOTE: For this work the fibers were manufactured in-house using a non-biodegradable proprietary polymer, NMP as the solvent and H₂O as the non-solvent. The fibers used in the system described here are 1.05 mm outer diameter with a 600-700 μm lumen diameter. The fibers are porous with pore diameters measuring 2.28 μm±1.5 μm (mean±standard deviation). This is designed to separate cells from the media feed in the fiber lumen, replicating vasculature of tissues. Fibers can also be purchased from membrane suppliers such as Pall.

### 2. Module Fabrication

NOTE: The modules used in this study are made from 1 mm thick borosilicate glass with 2 side ports (FIG. 2A). The fibers in the module described here has 3× fibers with a combined outer surface area of 6.54 cm² which is the equivalent of roughly half of a well of a 6-well plate.
2.1. Siliconize modules before first use by coating the inner surface with Sigmacote and allowing to dry in a fume hood. Autoclave (121 °C, 1 atm, 20 min) to increase the life of the treatment.
2.2. Using a scalpel cut 75 mm-long fibers and insert three fibers into each module leaving ~7 mm excess length at each end (FIG. 3A).
3.3. Place ~0.5 ml of silicone glue into a weighing boat. Use a P200 pipette tip to pick up a small amount of silicone and work the glue into the ends of the module around the fibers to form a 3-5 mm plug (FIG. 3B). Allow to dry for >3 hr.
3.4. Using a scalpel cut the silicone flush with the glass module ends (FIG. 3C).
3.5. Wrap a small amount (~4 layers) of polytetrafluoroethylene (PTFE) tape around one side port.

### 3. System Setup and Sterilization

NOTE: Pump tubing and modules with fibers are not autoclaved and are sterilized using 70% ethanol. It is recommended to calibrate the pump tubing with the pump to be used. The procedure below is carried out in a laminar flow hood.
3.1. Autoclave (as section 2.1) all autoclavable components prior to setup.
3.2. Setup
3.2.1. Place 10 ml 70% ethanol into the reservoir bottle and setup the reservoir bottle, Q-series cap, feed tube, pump and pump tubing as in FIG. 4.
3.2.2. Loosely place an end cap over the PTFE-taped side port. Slide the ends of the L/S16 module connectors over the module ends and free side port. Connect a 40 mm section of L/S13 tubing to the module connector nearest the capped side port as in FIG. 2B.
3.2.3. Connect the module to the pump tubing, ensuring to orientate the module so the capped side port is nearest the pump.
3.2.4. Connect the permeate line and retentate line to the module connectors and the L/S14 of the Y-connector on the reservoir bottle. Ensure that the setup resembles the schematics in FIG. 4.
3.3. Sterilization
3.3.1. Pump ethanol through the module at 800 μl/hr (267 μl/hr per fiber) or 30 ml/hr for enough time to treat unautoclaved components for >30 mins (e.g. 1 hr) (adjust times if other sterilizing methods are used [6]).
3.4. Wash
3.4.1. To wash the ethanol out of the system, first turn off the pump and drain the tubing. First, detach the pump tubing from the module adaptor tubing. Hold the module aloft to drain the ethanol out of the fibers and retentate line, back into the reservoir bottle. Remove the side port end cap from the module to drain the ethanol from the module itself, and the permeate line. Reattach the side port end cap. The inlet pipe to the reactor is disconnected and placed in an empty container and the pump is turned on to empty the line between the (now empty) reservoir and the reactor. Disconnect the inlet pipe to the reactor and place in an empty container and turn on the pump to empty the line between the (now empty) reservoir and the reactor. Alternatively, reverse the flow of media on the pump to drain the pump tubing and feed line of ethanol. Turn off the pump, and reattach the pump tubing to the module adaptor.
3.4.2. Unscrew the ethanol bottle from the lid and replace with a bottle containing 10 ml of cell growth medium (such as EMEM, GMEM, DMEM or RPMI) without serum [for example DMEM (without serum) at 30 ml/h for 1 hour]. Pump the medium through the system until the retentate line is full of media. Clamp the retentate line to force permeation of the media through the fibers to wash the module. Wash for ~2 hr.

Then a pre-treatment step is carried out using proliferation medium (DMEM+10% FBS+1% P/S). The wash medium is replaced with a reservoir containing 10 ml of proliferation medium (DMEM+10% FBS+1% P/S) and pumped at 30 ml/h overnight. The wash medium is replaced with a reservoir containing 10 ml of proliferation medium (DMEM+10% FBS+1% P/S) and pumped at 30 ml/h overnight.

### 4. Seeding

NOTE: The media and supplements used in this protocol should be those that are established for the desired cell type. Please refer to the literature, the European Collection of Cell Cultures (ECACC) and American Type Culture Collection (ATCC) for further information. Prior to this method cells should be maintained according to established protocols for the desired cell type. For this work the C2C12 cells were use and maintained according to the distributors recommendations (ATCC).

NOTE: For C2C12 cells a separate pre-treatment step may be carried out. The seeding protocol presented below also serves to pre-culture the module with cell culture medium prior to cell growth. Should a more extensive pre-culture be required then this should be carried out prior to seeding the module by draining the wash media from the system, replacing with growth media and permeating this through the module for few hours. See section 7.5.2.1 for details on replacing media in the system.
4.1. Prepare a single cell suspension by trypsinization according to established protocols for the desired cell type. A general protocol for a T75 culture is as follows:

NOTE: The number of cells to use in the seeding step should be empirically determined for your desired cell type. The bioreactors described here are seeded at a For C2C12 cells, the bioreactors are seeded at a 4-fold higher cell density that used in 2D tissue culture plastic for a 7 day culture.
4.1.1. Wash the cells by adding 10 ml phosphate buffered saline (PBS), aspirate, then add 3 ml 0.25% trypsin and ethylenediaminetetraacetic acid (EDTA) (enough to cover the cells) and incubate at 37 °C, 5% CO₂ for 5 min.
4.1.2. Harvest cells in 7 ml growth media supplemented with 10% fetal bovine serum (FBS) to neutralize the trypsin. Mix well, add 10 μl to the chamber of a hemocytometer and count the cells. A Chemometec NC-200 cell counter is used to perform the cell counts.
4.1.3. Centrifuge at 200×g for 5 min to pellet the cells.
4.1.4. Aspirate the supernatant and the C2C12 cell pellet obtained from a T-75 flask (at <80% confluence) is resuspended in 2 ml of media prior to cell count to ensure a high starting cell concentration.
4.2. Turn off the pump and drain the feed tube and module as in 3.4.1.
4.3. Detach the module from the module connectors and attach module end caps (Table 1) pre-sterilized in 70% ethanol, leaving one side port free.
4.4. Transfer 200 μl exactly of 160,500 cells (1.605×10\hat{ }5 cells) into the module (inoculation density of 25,000 cells/cm²). Then add additional proliferation media (no cells) to fill up any remaining space in the ECS of the module.

NOTE: It is important to use 200 μL in this example because when 500 μL is used there is insufficient ECS volume available, therefore not all of the cell suspension is transferred to the reactor.
4.1.1. Cap the side port using an end cap. Incubate the cells at 37 °C, 5% CO2 3 hours
4.1.1.1. The reactor module needs to be orientated and maintained at a set angle following the addition of the cell suspension, to prevent an uneven distribution of cells due to gravity settling. The module should remain at the selected angle for at least 10 minutes (specific to initial attachment time of C2C12s) prior to attaching to a tube rotator.
4.1.2. A tube rotator (Miltenyi Biotech MACSmixl) is used to rotate the modules intermittently at 12 rpm for 30 seconds with a 5 minute pause between rotation periods.
4.5. The modules can be drain using a 27G needle to introduce air or instead the side port end caps are removed and the module drained by gravity.
4.5.1 Following seeding, attach an end cap onto an injection port pre-sterilized in 70% ethanol, and attach this to the PTFE-taped side port. Remove the other side port end cap and slowly drain the cells by injecting air into the attached injection port using a 27 G needle and 1 ml syringe.
4.5.2. Replace the injection port with an end cap. Slowly fill the module with media using the free side port and an 18 G needle with a 1 ml syringe. Remove the module end caps and attach the module to the tubing using the module connectors.
4.6. Replace the wash media bottle with one containing 8.5 mL of proliferation media. Pump the growth media through the system.

NOTE: This is then replaced with a bottle containing differentiation medium (DMEM+2% horse serum+1% P/S) if differentiation is required.

### 5. Proliferation

NOTE: Continuous monitoring of dissolved oxygen concentration on the reactor inlet and outlet streams is carried out to provide on-line data of the proliferation of the C2C12 cells

NOTE: The fibers used in the research system described here are set to permeate at ~80 μl/hr, with an 800 μl/hr feed rate.
5.1. Use the fibers to grow the cells for periods of up to 6 days in a humidified incubator set at 37 °C, 5% CO2.

NOTE: Monitoring of nutrients and metabolites during the growth phase can provide useful information on the proliferation, metabolic uptake and output of the cells and nutrient and metabolite levels in the media. For example glucose usage and lactate production. A metabolic analyser (Roche CEDEX) may be used to measure the concentrations of glucose, lactate, glutamine and ammonia. Kits are available from various suppliers that can quantitate these factors from media (see Table 1 for those used in this study). Injection ports can be added to the permeate and retentate tubing and media sampled via a 27 G needle and syringe, and media can be sampled from the media reservoir bottle. This gives nutrient and metabolism information for the input and both output streams. Sampling should be carried out in a laminar flow hood. Sterilize injection ports before sampling by holding an ethanol soaked blue roll against the port for >30 sec.

### 6. Excision

NOTE: Fibers can be excised from the module at the end of an experiment for analysis.
6.1. Unplug and drain the HFB.
6.2. Insert a scalpel/micro knife blade between the glass and silicone. Turn the module so as to cut away the silicone from the glass. Repeat this procedure at both ends of the module.
6.3. Using the blade hook out the silicone plug from one end and gently pull. Ensure that the fibers come with it.

### 7. Cell Analysis

7.1. Cell numbers

NOTE: For the C2C12 cells used in this study any time points within the 6 day growth period are suitable for use in this calculation as growth rates do not substantially change over the cell densities achieved in this timeframe.
7.1.1. After excision (Section 6) dip the fibers into PBS to wash and cut them into a 1.5 ml tube containing 0.5 ml Tris EDTA (TE) buffer.
7.1.2 Cut the fibers into thirds along the axial length (to represent the inlet, central and outlet sections)
7.1.3 Cut into 1 ml of Trypsin (0.25%) and place in the incubator for 5 minutes.
7.1.4 Then use vigorous pipette mixing to agitate and help detach the cells from the fibers and break up cell aggregates.

7.1.5 Then place a chemometec NC-200 cassette into the suspension and take a cell sample to perform a cell count and cell viability check using the Chemometec NC-200 cell counter.
7.2. Cell proliferation rates
7.2.1. Using the cell numbers calculated at two different time points calculate the specific growth rate μ (Equation 1) where Ln(X1) is the natural log of the cell number at the first time point and Ln(X2) is the natural log of the cell number at the second time point.

μ=(Ln(X2)−Ln(X1))/time(hr) EQUATION 1:

From this calculate the population doubling times (dT) (Equation 2) where u is the specific growth rate.

dT=Ln2/μ EQUATION 2:
7.3. Cell viability
7.3.1. After excision (Section 6) dip the fibers into PBS to wash and cut them into a 1.5 ml tube containing 500 μl 0.05% trypsin ethylenediaminetetraacetic acid (EDTA). Incubate at 37 °C for 10 min.
7.3.2. Mix and add 10 μl of cell suspension to 10 μl trypan blue. Load 10 μl onto a haemocytometer and count the number of dead (blue) and alive cells.
7.4. Imaging
7.4.1. After excision dip the fibers into PBS to wash and use scissors to cut them into smaller lengths into a 24-well plate. Add 400 μl 4% paraformaldehyde (in PBS) and incubate at RT for 20 min.
7.4.2. Wash with PBS by pipetting 400 μl on and off. Repeat this step with fresh PBS.
7.4.3. Add 400 μl 4′,6-diamidino-2-phenylindole (DAPI) diluted in PBS to 34 pprox . . . 100 ng/ml and incubate at RT for 20 min. Protect from light.
7.4.4. Wash with PBS twice (as 7.3.2) and once with H2O. Add a fluorescence mounting medium to cover the fiber and image immediately to collect data before the samples dry (DAPI ex/em; 359/461 nm).
7.4.5. Take images at different focal planes and use ‘focus stacking’ software (e.g., stack focuser plugin for ImageJ, below) to make a composite image showing a greatly expanded depth of field. This is required as the fibers are not flat.
7.4.5.1. Download ImageJ (http://imageJ.nih.gov/ij/) and the ‘stack-focuser’ plugin (http://rsb.info.nih.gov/ij/plugins/stack-focuser.html).
7.4.5.2. In ImageJ open the images to be stacked. Then in the ‘Image’ menu go to ‘Stacks’-‘Images to Stack’. In the ‘Plugins’ menu go to ‘Stack Focuser’. Specify an n for nxn kernel. Trial and error with ‘n’ may be required in order to generate an image with little ‘noise’. Values between 11 & 77 tend to work well.

### Example 2—Hollow Fibre Bioreactors for the Production of Cell-Based Meat

### Material and Methods

Cell culture was performed with the immortalised murine myoblast cell line C2C12 as a model skeletal muscle cell line and primary, adult rat skeletal muscle cells, RSKMCs (Sigma-Aldrich, R150-05a). The C2C12s were cultured with DMEM (Sigma Aldrich D5796) supplemented with 10% v/v foetal bovine serum, FBS (Fisher Scientific Ltd, 11573397) and 1% v/v penicillin/streptomycin, P/S (5,000 U penicillin and 5 mg/mL streptomycin, Sigma-Aldrich P4458). The RSkMCs were cultured with Rat Skeletal Muscle Cell Growth Medium (Sigma-Aldrich, R151-500). During culture and bioreactor runs, cells were incubated at 5% CO₂ and 37 °C. Cell viability was assessed using trypan blue exclusion with 0.4% or 0.04% trypan blue solution (Sigma-Aldrich, T8154) and haemocytometer cell counts.

The hollow fibre bioreactor system consisted of a Watson-Marlow 205U peristaltic pump with orange-white colour coded pump tubing, silicone tubing (i.d. 0.8 mm, L/S 13 Platinum-Cured, Masterflex 96410) and a feed bottle with a Whatman Hepa-vent filtration unit (Sigma-Aldrich, WHA67235000). The bioreactor module was a custom-built glass module with two inlet/outlets and two side ports (Soham Scientific) with dimensions of i.d. 3=mm, o.d.=5 mm and L=60 mm. Optimisation of the system included the use of in-line dissolved oxygen sensors (PreSens flow through cell for oxygen, FTC—SU-PST3-S) in conjunction with PreSens Fibox 4 oxygen reader. The hollow fibres used in the system were initially 10% PLGA followed by porous, hydrophilic polystyrene hollow fibres produced in-house via dry-wet spinning, developed by Luetchford et al. 2018. [1] The move to polystyrene hollow fibres, referred to as PX40, was made after biocompatibility of RSkMCs was tested on flat sheet membranes and compared to the control of tissue culture plastic, TCP using a resazurin-based assay to determine metabolic activity, as a proxy for cell growth. Reactors were used with either 2 or 3 hollow fibres and a total feed flow rate of 800 μl h−1. Reactor setup and operation was performed via the method in Storm et al. 2016, with a 3 h attachment period and dynamic seeding using a MACSMix rotator. [2]

### Results

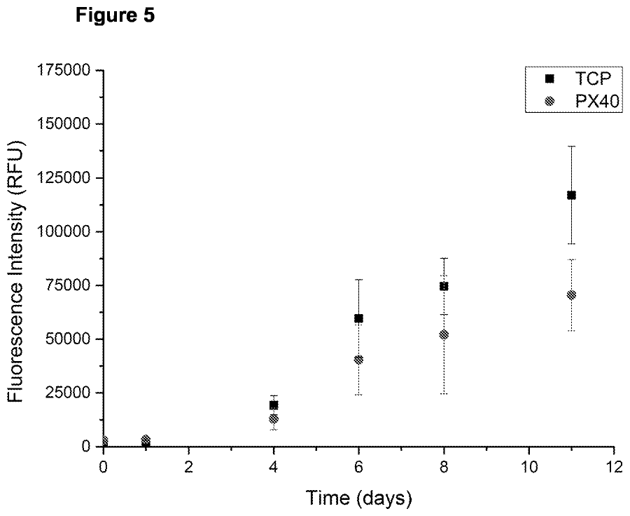

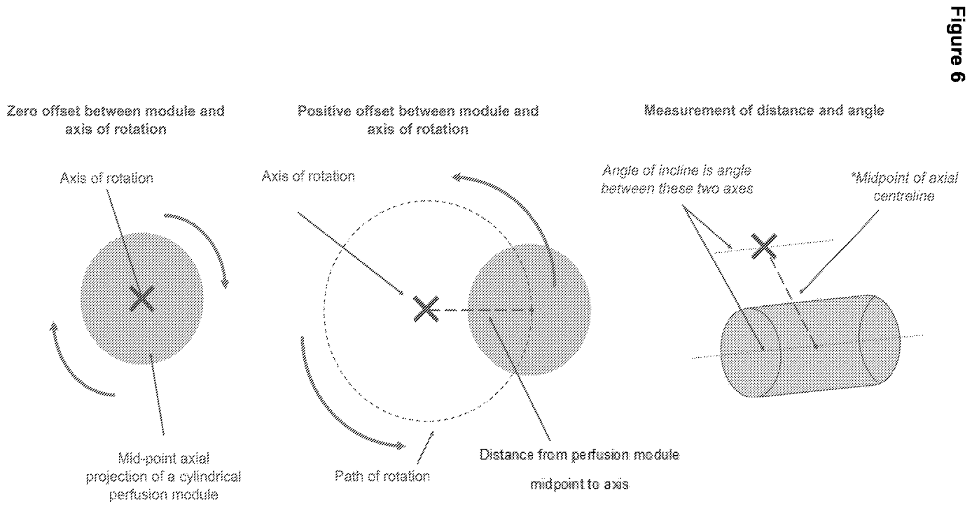

The results presented here summarise the work conducted in this study. Firstly, suitability of porous, hydrophilic polystyrene membranes (PX40) as a scaffold for cell growth was assessed in terms of biocompatibility and ability to sustain the growth of viable cells. This was carried out for RSkMC and compared against tissue culture plastic (TCP) as the positive control, as illustrated in FIG. 5.

The results of several bioreactor runs are presented in Table 1. The starting number of cells, N0 attached to the hollow fibres at day 0 is assumed to be 10% of the inoculum number due to cell seeding efficiency on hollow fibres. This efficiency is based on previous research in the Ellis group on MG-63s, a human osteosarcoma (bone) cell line. [3]

TABLE 1
Summary of cell numbers achieved after a given proliferation period within a hollow
fibre bioreactor. The inoculation cell number represents the number of cells placed
in the reactor at day 0, the seeded cell number (N0) is the starting cell number
and is based on an assumed seeding efficiency of 10% and NF is the final, total
number of cells present at the end of the run (viable and non-viable).

| | | | Run | | | | | |
|---|---|---|---|---|---|---|---|---|
| | Cell | Fibre | time | Inoculum | N0 | NF | SD | Viability |
| Run | line | type | (days) | (cells) | (cells) | (cells) | (cells) | (%) |
| 1 | C2C12 | PLGA | — ^{a} | — | — | — | — | — |
| 2 | C2C12 | PLGA | 7 ^{b} | 7.29 × 10⁵ | 7.29 × 10⁴ | 7.67 × 10^{5 c} | 1.83 × 10⁵ | — ^{c} |
| 3 | RSKMC | PLGA | 7 | 7.56 × 10⁵ | 7.56 × 10⁴ | 1.18 × 10^{6 c} | 2.33 × 10⁵ | — ^{c} |
| 4.1 | RSKMC | PX40 | 6 | 1.20 × 10⁶ | 1.20 × 10⁵ | 1.02 × 10^{6 c} | 1.45 × 10⁵ | — ^{c} |
| 4.2 | RSKMC | PX40 | 6 | 1.20 × 10⁶ | 1.20 × 10⁵ | 9.48 × 10^{5 c} | 1.98 × 10⁵ | — ^{c} |

PLGA = poly(lactic-co-glycolic acid, PX40 = polystyrene based polymer with 40% w/w microcrystalline sodium chloride.
^{a} Unsuccessful run, HFB module ran dry (cells not exposed to nutrient supply), suspected back pressure issues;
^{b} Pump stopped at day 3 due to power outage (4 days of no media circulation followed); and
^{c} Trypan blue exclusion assay during cell counts indicated cells were non-viable.

### Discussion

The use of the polystyrene membranes as the scaffold was deemed suitable following the initial biocompatibility assay (FIG. 5) of RSkMCs on 2D flat sheet membranes. FIG. 5 shows that the cells are metabolically active and consequently able to attach to and grow on the scaffold. Polystyrene is non-edible and non-biodegradable; therefore the polystyrene scaffold presents the potential opportunity to be re-used for several batches to minimize the operating expenses associated with skeletal muscle cell expansion.

The initial reactor studies that were not subject to unforeseen equipment malfunctions and consequently successfully housed cells for the desired period of time, with medium perfusion, are reactor runs 3, 4.1 and 4.2. By taking into account the assumption of a 10% seeding efficiency on hollow fibres, it can be seen from Table 1 that cell proliferation occurred in all reactor runs as NF>N0. After each of these runs the viability assessment using trypan blue exclusion indicated that all the cells were non-viable. It is suspected that these may be false negative results resulting from the sensitivity of the primary cell line, RSkMC to trypan blue or the impact of exposure to the dissociation enzyme trypsin. The suspected cause of over-exposure to trypan blue leading to false non-viable cell identification may be a result of the high concentration of 0.4% or the period of exposure before cell counts, these support the inconclusive colour of the cells during analysis.

The number of cells obtained from the bioreactor systems can theoretically be increased by increasing the duration of the proliferation phase and time per reactor run and the number of hollow fibres within the reactor module. The number of hollow fibres dictates the surface area available for cell attachment and growth and a confluent surface dictates the need for transferring to a larger reactor system, known as passaging. The NF values are suspected to be an under-estimate of the total, final number of cells due to the presence of large cell aggregates during haemocytometer counting. The presence of aggregates is suspected to result from the method of cell dissociation from the fibre scaffolds, trypsin is currently used.

Future work will involve the expansion of RSKMCs in the bioreactor system with instrumentation for on-line monitoring to verify their viability. Use of a DNA quantification method such as the PicoGreen assay to quantify cell numbers as an alternative to manual cell counting. Scaling-up of expansion through longer proliferation periods, the incorporation of more fibres to increase available scaffold surface area and the use of larger reactor modules or numbering up by operating multiple HFBs run in parallel.

### Conclusions

This study satisfies the initial aims of this work and provides a proof of concept for the use of hollow fibres, specifically porous polystyrene (PX40) fibres, in hollow fibre bioreactors for skeletal muscle cell attachment and proliferation. For the immortalized murine myoblast cell line, C2C12s confirmation that cells were alive with a viability of c.a. 80% after a 5-day culture was presented based on trypan blue assessment.

### Example 3—Cell Attachment Assays

### Protocol Outline

The desired cell number (suspension concentration of cells per mL and the corresponding volume in mL to reach target seeding density). A suspension volume less than or equal to the ECS volume was first injected into the reactor and then topped up with fresh media to avoid losses of cells if not all of the ECS volume was not injected into side port of reactor from the original seeding suspension volume.

The module was mounted in a specially designed saddle clip on rotor at specific angle (in this case, 9.2 degrees).

Constant rotation was applied to ensure good distribution of cells across the fibre bundle.

The module was removed; unattached cells removed by draining the growth medium from the side port and replaced with fresh growth medium and connected to a continuous, dynamic feeding system following seeding.

The seeding regime comprised of rotating the perfusion module inclined at 9.2 degrees, rotating continuously at 0.3 RPM, the module was offset by 150 mm from the axis of rotation.

Viability of cells was determined by either a direct or indirect method of cell viability measurement.

Direct measurement—cells were seeded at required density and allowed to attached over given period. Cells were recovered from excised fibres and quantification via direct cell count (Chemometec NC-200) to give VCC (viable cell count).

Indirect measurement—cells were seeded at required density and allowed to attached over given period. The module was drained in order to replace growth medium. Cells contained in the drained growth medium were quantified as total cell count (non-viable included). Indirect attachment efficiency=(inoculated cells-removed during drain post-seed)/inoculated cells (as %).

### Results

Increasing time improves initial attachment strength which manifests in better attachment efficiency when quantified directly* (See “Test Objective—Attachment Efficiency” experiments) or indirectly** (all other experiments). Cells lay down a layer of ECM gradually meaning looser attachment at the start of attachment—the attachment strength is therefore improved.

### Discussion

The dataset shows for the attachment efficiency experiments, there is a range of values from 35.4% to 71.4%. These measurements are obtained through direct measurement of cells removed using cell removal agent and quantified via direct cell count (Chemometec NC-200) to give VCC (viable cell count).

TABLE 2
Cell attachment quantification

| | | | | Charac- | | | | |
|---|---|---|---|---|---|---|---|---|
| | | | | teristic | | | | |
| | | | | distance | | | | |
| | | | | from | Total | | | |
| | | | | module | internal | | | Inoculum, |
| | | | Angle of | midpoint to | volume of | | | N0 |
| | Test | | incline | axis of | reactor | No. | SA | (viable - |
| HFB ID | Objective ^{A} | RPM | (degrees) | rotation/mm | (mL) | fibres | (cm²) ^{B} | cells) ^{C} |
| HFB0043 | Prolif- | 0.3 | 9.2 | 150 | 17 | 39 | 205.82 | 8.16E+06 |
| | eration | | | | | | | |
| HFB0044 | Prolif- | 0.3 | 9.2 | 150 | 17 | 40 | 186.68 | 5.76E+06 |
| | eration | | | | | | | |
| HFB0045 | Attachment | 0.3 | 9.2 | 150 | 17 | 40 | 186.68 | 5.76E+06 |
| | efficiency | | | | | | | |
| HFB0046 | Prolif- | 0.3 | 9.2 | 150 | 17 | 40 | 186.68 | 5.76E+06 |
| | eration | | | | | | | |
| HFB0047 | Prolif- | 0.3 | 9.2 | 150 | 17 | 40 | 186.68 | 5.76E+06 |
| | eration | | | | | | | |
| HFB0048 | Prolif- | 0.3 | 9.2 | 150 | 17 | 40 | 192.84 | 4.30E+06 |
| | eration | | | | | | | |
| HFB0049 | Attachment | 0.3 | 9.2 | 150 | 17 | 40 | 192.84 | 4.30E+06 |
| | efficiency | | | | | | | |
| HFB0050 | Prolif- | 0.3 | 9.2 | 150 | 17 | 40 | 192.84 | 4.30E+06 |
| | eration | | | | | | | |
| HFB0051 | Control | 0.3 | 9.2 | 150 | 17 | 40 | 192.84 | 4.30E+06 |
| | loop | | | | | | | |
| | [prolif- | | | | | | | |
| | eration] | | | | | | | |
| HFB0056 | Attachment | 0.3 | 9.2 | 150 | 17 | 39 | 188.02 | 4.41E+06 |
| | efficiency | | | | | | | |
| HFB0057 | Attachment | 0.3 | 9.2 | 150 | 17 | 40 | 192.84 | 4.64E+06 |
| | efficiency | | | | | | | |
| HFB0058 | Differen- | 0.3 | 9.2 | 150 | 17 | 40 | 192.84 | 8.73E+06 |
| | tiation | | | | | | | |
| HFB0059 | Differen- | 0.3 | 9.2 | 150 | 17 | 40 | 192.84 | 8.73E+06 |
| | tiation | | | | | | | |
| HFB0052 | Prolif- | 0.3 | 9.2 | 150 | 289 | 300 | 2991.42 | 2.22E+08 |
| | eration | | | | | | | |
| HFB0053 | Prolif- | 0.3 | 9.2 | 150 | 289 | 297 | 2832.75 | 2.22E+08 |
| | eration | | | | | | | |
| HFB0054 | Differen- | 0.3 | 9.2 | 150 | 289 | 321 | 3154.44 | 2.22E+08 |
| | tiation | | | | | | | |
| HFB0055 | Differen- | 0.3 | 9.2 | 150 | 289 | 287 | 3152.12 | 2.96E+08 |
| | tiation | | | | | | | |
| HFB0060 | Differen- | 0.3 | 9.2 | 150 | 289 | 302 | 2967.72 | 2.83E+08 |
| | tiation | | | | | | | |
| HFB0061 | Differen- | 0.3 | 9.2 | 150 | 289 | 298 | 3272.94 | 3.12E+08 |
| | tiation | | | | | | | |

| | | | Post- | Attachment | Attachment |
|---|---|---|---|---|---|
| | Inoculum | | seed | efficiency | efficiency, |
| | density, | | unattached | LOST CELL | DIRECT |
| | X0 | | cells | RECOVERY | EXCISED |
| | (viable - | Attachment | (total - | ESTIMATE (%) | FIBRE (%) |
| HFB ID | cells/cm2) ^{D} | period (h) | cells) ^{E} | INDIRECT | DIRECT |
| HFB0043 | 39,645 | 3.82 | 1.33E+06 | 84% | — |
| HFB0044 | 30,855 | 4.05 | 3.55E+06 | 38% | — |
| HFB0045 | 30,855 | 4.05 | 2.58E+06 | 55% | 42.0% |
| HFB0046 | 30,855 | 4.05 | 1.38E+06 | 76% | — |
| HFB0047 | 30,855 | 4.05 | 1.85E+06 | 68% | — |
| HFB0048 | 22,298 | 3.50 | 2.73E+05 | 94% | — |
| HFB0049 | 22,298 | 5.00 | 5.31E+05 | 88% | 35.4% |
| HFB0050 | 22,298 | 3.50 | 2.75E+05 | 94% | — |
| HFB0051 | 22,298 | 3.50 | 5.66E+05 | 87% | — |
| HFB0056 | 23,455 | 5.05 | 1.39E+05 | 97% | 71.4% |
| HFB0057 | 24,062 | 5.05 | 2.29E+05 | 95% | 56.2% |
| HFB0058 | 45,271 | 3.92 | 3.39E+05 | 96% | — |
| HFB0059 | 45,271 | 4.42 | 4.62E+05 | 95% | — |
| HFB0052 | 74,078 | 3.83 | 6.63E+06 | 97% | — |
| HFB0053 | 78,228 | 5.00 | 6.31E+06 | 97% | — |
| HFB0054 | 70,250 | 4.17 | 4.00E+07 | 82% | — |
| HFB0055 | 93,778 | 4.17 | 1.53E+07 | 95% | — |
| HFB0060 | 95,359 | 4.25 | 1.54E+07 | 95% | — |
| HFB0061 | 95,327 | 4.08 | 2.94E+07 | 91% | — |

^{A} Proliferation experiments are those which are run for 5-7 days. The cells are not removed from the fibres directly after seeding, hence only an indirect measurement was used.
^{B} The calculated surface area of fibres based on fibre diameter, module length and number of fibres. Allows for calculation of inoculum - number of cells used in cell seed suspension.
^{C} Quality check of number of viable cells inoculum.
^{D} The viable cell density per area placed in to the reactor before the seeding process.
^{E} The number of unattached cells, allowing for calculation of cell attachment efficiency by indirect measurement.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### REFERENCES

[1] K. A. Luetchford et al., “Next generation in vitro liver model design: Combining a permeable polystyrene membrane with a transdifferentiated cell line,” J. Memb. Sci., vol. 565, pp. 425-438, November 2018.
[2] M. P. Storm et al., “Hollow Fiber Bioreactors for In Vivo-like Mammalian Tissue Culture,” J. Vis. Exp., no. 111, pp. 1-12, 2016.
[3] S. M. Acott, “Cell Expansion And Delivery Methods For Heart Regeneration [Thesis],” University of Bath, 2016.
[4]. Mulder, M. The basic principles of membrane technology. 2nd ed. Chapter 3 section 4. Kluwer Academic Publishers, (1996).
[5]. Ellis, M. J., Chaudhuri, J. B. Poly(lactic-co-glycolic acid) hollow fibre membranes for use as a tissue engineering scaffold. Biotechnol Bioeng. 96 (1), 177-187 (2007).
[6]. Shearer, H., Ellis, M. J., Perera, S. P., Chaudhuri, J. B. Effects of common sterilization methods on the structure and properties of poly(D,L lactic-co-glycolic acid) scaffolds. Tissue Eng. 12 (10), 2717-2727 (2006).

1. A method of culturing muscles cells for a comestible product, the method comprising:
a) providing a perfusion module for a perfusion bioreactor system;
the perfusion module comprising one or more porous hollow fibres comprising an outer surface and an internal lumen;
b) seeding muscle cells onto the one or more porous hollow fibres;
c) maintaining the perfusion module in an orientation wherein the one or more porous hollow fibres is at an angle between 0 to 90 from horizontal for a time period sufficient to allow initial attachment of the muscle cells to an outer surface of each of the one or more porous hollow fibres;
d) rotating the perfusion module;
e) connecting the perfusion module to a perfusion bioreactor system; and
f) culturing the muscle cells attached to the outer surface of each of the one or more porous hollow fibres under conditions suitable for proliferation and/or differentiation of the muscle cells.

2. The method of claim 1, wherein the time period is at least 10 minutes.

3. The method of claim 1 or 2, wherein the angle is between 0 and 30 degrees from horizontal; preferably between 5 and 20 degrees; more preferably between 8 and 10 degrees.

4. The method of claims 1 to 3, wherein rotating comprises exerting a centrifugal force of between 0 and 50 N.

5. The method of any one of claims 1 to 4, wherein rotating comprises continuously rotating; optionally at a speed of between 0 and 30 rpm.

6. The method of claims 1 to 4, wherein rotating the perfusion module comprises intermittently rotating the perfusion module.

7. The method of claim 6, wherein rotating the perfusion module comprises rotating at a speed of between 0 and 30 rpm for about 1 second to about 5 minutes every 10 seconds to about 30 minutes for a total time of at least 1 hour; optionally at a speed of about 12 rpm for about 30 seconds about every 5 minutes.

8. The method of any preceding claim, wherein rotating the perfusion module comprises rotating for at least three hours; optionally about 3 to 5 hours.

9. The method of any preceding claim, wherein rotating the perfusion module comprises rotating the perfusion module with an offset between the mid-point of the centreline of the perfusion module and the axis of rotation; optionally wherein the offset is at least 0.01 mm.

10. The method of any preceding claim, wherein the attachment efficiency is at least 55% as measured by quantification of viable cells attached to the hollow fibres by direct or indirect measurement.

11. The method of any preceding claim, wherein prior to seeding the muscle cells are:
(i) washed with a composition comprising a cell detachment agent;
(ii) harvested in a culture media comprising a growth promotion agent;
(iii) centrifuged to form a pellet; and
(iv) resuspended in a culture media to form a cell suspension.

12. The method of claim 11, wherein seeding the muscle cells comprises applying the cell suspension comprising the muscle cells to the perfusion module.

13. The method of any preceding claims, wherein seeding the muscle cells comprises applying around a 0.1 to 10-fold higher cell density than used for 2D tissue culture plastic.

14. The method of any preceding claims, wherein seeding the muscles cells comprises applying at least 100 cells/cm² to the perfusion module.

15. The method of any preceding claim, wherein the one or more porous hollow fibres:
are hydrophilic;
are washable; and/or
are reusable.

16. The method of any preceding claim, wherein the one or more porous hollow fibres comprise polystyrene.

17. The method of any one of claims 1 to 15, wherein the one or more porous hollow fibres are biodegradable and/or edible.

18. The method of any preceding claim, wherein culturing comprises pumping culture media through the perfusion bioreactor system and the internal lumen of each of the one or more porous hollow fibres; optionally
wherein culturing further comprises maintaining the perfusion module is maintained in an orientation wherein the one or more porous hollow fibres is at an angle between 0 to 90 from horizontal; optionally wherein the angle is between 0 and 30 degrees from horizontal; preferably between 5 and 20 degrees; more preferably between 8 and 10 degrees.

19. The method of any preceding claim, wherein culturing comprises pumping culture media through the perfusion bioreactor system and the internal lumen of each of the one or more porous hollow fibres at a rate of at least 10 μL/hour/hollow fibre.

20. The method of any preceding claim, wherein culturing comprises maintaining the muscle cells at a temperature of at least 15 °C and/or 5% CO₂.

21. The method of any preceding claim, wherein culturing comprises pumping culture media through the perfusion bioreactor system and perfusion module for at least 3 hours.

22. The method of any preceding claim, wherein culturing comprises pumping a first culture media through the perfusion bioreactor system and the internal lumen of each of the one or more porous hollow fibres, optionally wherein the first culture media is a proliferation medium.

23. The method of claim 22, wherein culturing further comprises pumping a second culture media through the perfusion bioreactor system and the internal lumen of each of the one or more porous hollow fibres, optionally wherein the second culture media is a differentiation medium; further optionally wherein the differentiation media is perfused through the bioreactor system and perfusion module for at least 3 hours.

24. The method of any preceding claim, wherein the perfusion bioreactor system comprises apparatus for monitoring metabolite concentration and/or oxygen concentration of the culture media.

25. The method of any preceding, wherein the method further comprises:
(g) harvesting the muscle cells from the perfusion module.

26. The method of claim 25, wherein the harvested muscle cells are formed into a cultured meat product; or
wherein the one or more hollow fibres are edible, and the harvested cells and the one or more porous hollow fibres are formed into a cultured meat product.

27. The method of any preceding claim, wherein the muscle cells are derived from at least one comestible animal cell; optionally wherein the muscle cells comprise one or more of fibroblasts, skeletal muscle cells, smooth muscle cells, and/or myoblasts; further optionally wherein the muscle cells ae derived from one or more of non-human embryonic stem cells and/or pluripotent stem cells.

28. A comestible product comprising muscle cells obtained by the method according to any one of claims 1 to 27.

29. A comestible product obtainable by the method according to any one of claims 26 and 27.

30. The method of any one of claims 1 to 27, or the comestible product of claim 28 or 29, wherein the comestible product is a cultured meat product.
